SEQUENCE LISTING (11 sequences)

SEQ ID NO: 1 - protein, 5 aa, Artificial Sequence
  CDR
ETYIH

SEQ ID NO: 2 - protein, 17 aa, Artificial Sequence
  CDR
RIDPADVYGRYDPKFQG

SEQ ID NO: 3 - protein, 10 aa, Artificial Sequence
  CDR
SYGSSYGIDY

SEQ ID NO: 4 - protein, 11 aa, Artificial Sequence
  CDR
RASQDISNYLN

SEQ ID NO: 5 - protein, 7 aa, Artificial Sequence
  CDR
YTSTLHS

SEQ ID NO: 6 - protein, 9 aa, Artificial Sequence
  CDR
QQGYTLPWT

SEQ ID NO: 7 - protein, 462 aa, Artificial Sequence
  Heavy Chain
MKCSWVIFFLMAVVTGVNSEVQLQQSGAELVKPGASVKLSCTASGFNIKETYIHWVKQRPEQGLEWIGRIDPADVYGRYDPKFQGKATITADTSSNSAYLQVSSLTSEDTAVYYCARSYGSSYGIDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 8 - protein, 234 aa, Artificial Sequence
  Light Chain
MMSSAQFLGLLLLCFQGTRCDIQMTQTTSSLSASLGDRVTISCRASQDISNYLNWYQQKPDGTVKLLIYYTSTLHSGVPSRFSGSGSGTDYSLTISNLEQEDVATYFCQQGYTLPWTFGGGTKLEIKRADAAPTVSIFPPSSEQLTSGGASVVCFLNNFYPKDVNVKWKIDGSERQSGVLNSWTDQDSKDSTYSMSSTLTLTKDEYERHNSYTCEATHKTSTSPIVTSFNRNEC

SEQ ID NO: 9 - DNA, 1389 bp, Artificial Sequence
  Heavy Chain
atgaaatgcagctgggttatcttcttcctgatggcagtggttacaggggtcaattcagaggttcagctgcagcagtctggggcagagcttgtgaagccaggggcctcagtcaagttgtcctgcacagcttctggcttcaacattaaagaaacctatatacactgggtaaaacagaggcctgaacagggcctggagtggattggaaggattgatcctgcggatgtttatggtagatatgacccgaagttccagggcaaggccactataacagcagacacatcctccaattcagcctacctgcaggtcagcagcctgacatctgaggacactgccgtctattactgtgctagatcctacggtagtagctatggtattgactactggggtcaaggaacctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 10 - DNA, 705 bp, Artificial Sequence
  Light Chain
atgatgtcctctgctcagttccttggtctcctgttgctctgttttcaaggtaccagatgtgatatccagatgacacagactacatcctccctgtctgcctctctgggagacagagtcaccatcagttgcagggcaagtcaggacattagcaattatttaaactggtatcagcaaaaaccagatggaactgttaaactcctgatctactacacatcaacattacactcaggagtcccatcaaggttcagtggcagtgggtctggaacagattattctctcaccattagcaacctggagcaagaagatgttgccacttacttttgccaacagggttatacacttccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacgtcaatgtcaagtggaagattgatggcagtgaacgacaaagtggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcacgagcttcaacaggaatgagtgttag

SEQ ID NO: 11 - protein, 241 aa, Homo sapiens
MAVFLQLLPLLLSRAQGNPGASLDGRPGDRVNLSCGGVSHPIRWVWAPSFPACKGLSKGRRPILWASSSGTPTVPPLQPFVGRLRSLDSGIRRLELLLSAGDSGTFFCKGRHEDESRTVLHVLGDRTYCKAPGPTHGSVYPQLLIPLLGAGLVLGLGALGLVWWLHRRLPPQPIRPLPRFAPLVKTEPQRPVKEEEPKIPGDLDQEPSLLYADLDHLALSRPRRLSTADPADASTIYAVVV

(19) United States
(12) Patent Application Publication
(10) Pub. No.: US 2022/0112285 A1
(43) Pub. Date: Apr. 14, 2022

(54) ANTIGEN BINDING MOLECULES AND EPITOPES, AND USES THEREOF

(71) Applicant: THE UNIVERSITY OF BIRMINGHAM, Birmingham (GB)

(72) Inventors: Peter Yotis SENIS, Birmingham (GB); Alexandra MAZHARIAN, Birmingham (GB); Timo VÖGTLE, Birmingham (GB)

(21) Appl. No.: 17/429,580

(22) PCT Filed: Feb. 26, 2020
(86) PCT No.: PCT/GB2020/050456

§ 371 Date: Aug. 9, 2021

(30) Foreign Application Priority Data
Feb. 26, 2019 (GB) 1902590.7

(51) Int. Cl. C07K 16/28; A61P 7/04; A61K 45/06; A61K 39/395

(52) U.S. Cl. CPC C07K 16/2803; A61P 7/04; A61K 2039/505; A61K 39/3955; A61K 45/06

(57) ABSTRACT

The invention provides an isolated antigenbinding molecule or fragment thereof which specifically binds to human G6b-B. The invention also provides for use of the antigen binding molecule or fragment thereof in treating a disease or disorder associated with dysregulated platelet homeostasis.

The present invention relates to novel antigen binding molecules and epitopes, and in particular to antibodies which specifically target epitopes on the G6b-B receptor. Such antigen binding molecules may be used to modulate platelet and/or megakaryocyte function.

Platelets are small fragments of megakaryocytes (MKs) that play a critical role in thrombosis, hemostasis and maintenance of vascular function. They do so by adhering to exposed extracellular matrix proteins at sites of vascular injury, where they become activated and form a hemostatic plug, preventing excessive blood loss and stimulating wound repair. The mechanisms required to maintain hemostasis also facilitate the formation of occlusive thrombi, which can lead to ischemia in acute coronary heart disease and stroke, two of the leading causes of death worldwide.

Immunoreceptor tyrosine-based inhibition motif (ITIM)-containing receptors are involved in the control of a broad spectrum of biological functions. ITIMs become phosphorylated on the central tyrosine residue embedded in the consensus sequence ([I/V/L]xYxx[V/L]) by Src family kinases (SFKs) and subsequently recruit phosphatases, most notably phosphatidylinositol 3,4,5-trisphosphate 5-phosphatase 1 (SHIP1) and the SH2 domain-containing protein-tyrosine phosphatases (Shp)1 and 2. Despite strong similarities in intracellular signalling pathways, there is no pattern for the ligands for ITIM-containing receptors, and the physiological ligands of many of these receptors are not known.

Unique among platelet ITIM-containing receptors is G6b-B, which is highly expressed in mature MKs and platelets (Coxon et al., 2017, Blood, 129(26):3407-3418; Senis et al., 2007, Mol. Cell Proteomics, 6(3):548-564). G6b-B is a type I transmembrane protein that consists of a single N-glycosylated immunoglobulin-variable (IgV)-like domain in its extracellular region, a single transmembrane domain and a cytoplasmic tail containing an ITIM and an immunoreceptor tyrosine-based switch motif (ITSM), which provide a high affinity docking site for Shp1 and Shp2 upon phosphorylation. The inhibitory function of G6b-B has been demonstrated in a heterologous cell system and by antibody-mediated crosslinking of the receptor in platelets, which resulted in attenuated platelet aggregation and activation (Mori et al., 2008, J. Biol. Chem., 283(51):35419-27). Importantly, the generation and analysis of G6b-B knockout (KO) mice demonstrated that the function of G6b-B goes beyond inhibiting signalling from ITIM-containing receptors (Mazharian et al., 2013, Blood, 121(20):4205-4220). G6b-B knockout mice develop a severe macrothrombocytopenia and aberrant platelet function, establishing G6b-B as a critical regulator of platelet activation and production. This phenotype was also observed in a G6b-B loss-of-function mouse model in which the tyrosine residues within the ITIM and ITSM were mutated to phenylalanine residues, abrogating binding of Shp1 and Shp2 to G6b-B and downstream signalling (Geer et al., 2018, Blood, 132(13):1413-1425). Moreover, expression of human G6b-B in mouse platelets rescued the phenotype of G6b-B-deficient mice, demonstrating that human and mouse G6b-B exerts the same physiological functions (Hofmann et al., 2018, Blood, 132(13):1399-1412). Importantly, null and loss-of-function mutations in human G6b-B have been reported to recapitulate key features of the G6b-B KO and loss-of-function mouse phenotypes, including a severe macrothrombocytopenia, MK clusters in the bone marrow and myelofibrosis (Hofmann et al., 2018, Blood, 132(13):1399-1412; Melhem et al., 2016, Eur J Haematol., 98(3):218-227).

Bleeding and thrombosis are major causes of morbidity and mortality in patients with chronic myeloproliferative neoplasms (MPN), yet current therapies are limited, with high risk of relapse and severe side-effects. Current therapies of MPN include tyrosine kinase inhibitors (Gleevec and Jak inhibitors), steroids, chemotherapy and radiation therapy, blood transfusion and bone marrow replacement. All of these therapies come with moderate to severe side-effects, with little or no effect on associated myelofibrosis, haemorrhagic and thrombotic complications. MPN can lead to increased megakaryocyte (MK) counts in sites of haematopoiesis, myelofibrosis, platelet production and aberrant platelet function, predisposing patients to bone marrow (BM) failure, bleeding and thrombotic complications.

Given the critical role of platelets in hemostasis and diseases and disorders caused as a result in breakdown of platelet homeostasis (including a change in the level of platelet production and/or a change in platelet reactivity), and the lack of effective treatments for these, there is a need to provide new alternative ways to treat diseases and disorders associated with platelet homeostasis.

The invention provides novel antigen binding molecules which specifically bind a newly-identified epitope in the C-terminus of the ectodomain of G6b-B. Preferably these antigen binding molecules do not interfere with ligand binding, preferably heparin binding, and/or dimerization of G6b-B. The antigen binding molecule may facilitate clustering of ligand-induced G6b-B dimers and may inhibit downstream G6b-B signalling, therefore altering platelets to become more reactive to classical agonists, such as collagen, and in addition or alternatively regulating platelet production.

An antigen binding molecule of the invention may bind to the herein defined epitope in the C-terminus of the ectodomain of G6b-B, and inhibit inhibitory signalling from G6b-B by altering the clustering of heparin ligand-induced G6b-B dimers. This may result in increased signalling from activatory receptors that G6b-B normally suppresses, thus altering platelet reactivity and/or platelet production in the bone marrow. Treatment of humanized G6b-B mice with an antigen binding molecule of the invention results in a rapid, significant and sustained reduction in platelet counts. Furthermore, treatment of human platelets with an antigen binding molecule of the invention enhances the platelet reactivity to the classical agonist collagen in the presence of the G6b-B ligand heparin. Collectively, the inventors demonstrate that antigen binding molecules of the invention have significant biological effects on platelets and their function.

An aim of the present invention is therefore to provide antigen binding molecules which bind to a specific epitope on human G6b-B (as defined by Uniprot accession number: 095866 and the sequence in FIG. 14). A further aim is to provide antigen binding molecules which modulate platelet homeostasis for the treatment of diseases or disorders associated with dysregulated platelet homeostasis, such as dysregulated platelet production or reactivity, for example in myeloproliferative or thrombotic diseases and disorders, for example macrothrombocytopenia, microthrombocytopenia and normothrombocytopenia.

In a first aspect, the invention provides an isolated antigen binding molecule or fragment thereof which specifically binds to human G6b-B.

In another aspect, there is provided an isolated antigen binding molecule or fragment thereof which competes for binding to G6b-B with an antigen binding molecule of the invention.

In an aspect, the isolated antigen binding molecule or fragment thereof may specifically bind to an epitope on human G6b-B defined by at least residues equivalent to Asp 24, Arg 26 and Gly 124 of the sequence of FIG. 14. In an embodiment, the antigen binding molecule or fragment thereof specifically binds to an epitope on human G6b-B defined by at least residues equivalent to Pro 19 to Arg26 and His121 to Gly124 of the sequence of FIG. 14. In an embodiment, the antigen binding molecule or fragment thereof binds to an epitope on human G6b-B defined by at least one or more, two or more, three or more of residues equivalent to Arg26, Asp 24, Gly25, His121, Val122, Leu123, Gly124, Asp125, Ser22, Asp29, Leu23, Val131, Gly20, Asp32, Ala21 and Pro19 of the sequence of FIG. 14, preferably the epitope includes at least residues equivalent to Asp 24, Arg 26 and Gly 124.

The isolated antigen binding molecule or fragment thereof of any aspect of the invention may be an antibody or a fragment thereof. In an embodiment, the antibody or fragment thereof is an isolated monoclonal antibody, bispecific antibody, ScFv, Fab, (Fab′)2, Fv, dAb, Fd or a diabody. Preferably, the antigen binding molecule is an isolated monoclonal antibody.

In another aspect, the isolated antigen binding molecule or fragment thereof comprises an antigen binding portion which comprises heavy and/or light chain variable regions which comprise one or more of the CDRs defined by Seq ID No: 1, 2, 3, 4, 5 and 6, or a sequence having at least 80%, 90%, 95%, 98%, 99% or 100% identity thereto, or a variant of one or more of the CDR sequences defined by Seq ID No: 1, 2, 3, 4, 5 and 6 having one, two or three amino acid variations from the recited CDR sequence provided that the antigen binding molecule or fragment thereof retains the ability to bind to G6b-B.

In another aspect, the isolated antigen binding molecule or fragment thereof may comprise an antigen binding portion comprising a heavy and/or light chain variable region, wherein:

a) the heavy chain variable region comprises one or more of the hypervariable regions comprising sequences of at least 80%, 90%, 95%, 98%, 99% or 100% identity to:
CDR1 of SEQ ID NO: 1
CDR2 of SEQ ID NO: 2
CDR3 of SEQ ID NO: 3; and/or

b) the light chain variable region comprises one or more of the hypervariable regions comprising sequences of at least 80%, 90%, 95%, 98%, 99% or 100% identity to:
CDR1 of SEQ ID NO: 4
CDR2 of SEQ ID NO: 5
CDR3 of SEQ ID NO: 6 or direct CDR equivalents thereof.

In an embodiment, the isolated antigen binding molecule or fragment thereof comprises an antigen binding portion comprising a heavy and/or light chain variable region, wherein:

a) the heavy chain variable region comprises the hypervariable regions:
CDR1 of SEQ ID NO: 1
CDR2 of SEQ ID NO: 2
CDR3 of SEQ ID NO: 3

or sequences of at least 80%, 90%, 95%, 98%, 99% or 100% identity thereto;

and/or

b) the light chain variable region comprises the hypervariable regions:
CDR1 of SEQ ID NO: 4
CDR2 of SEQ ID NO: 5
CDR3 of SEQ ID NO: 6

or sequences of at least 80%, 90%, 95%, 98%, 99% or 100% identity thereto.

In an embodiment, the isolated antigen binding molecule or fragment thereof comprises an antigen binding portion comprising a heavy and/or light chain variable region, wherein:

a) the heavy chain variable region comprises the hypervariable regions:
CDR1 of SEQ ID NO: 1
CDR2 of SEQ ID NO: 2
CDR3 of SEQ ID NO: 3;

and

b) the light chain variable region comprises the hypervariable regions:
CDR1 of SEQ ID NO: 4
CDR2 of SEQ ID NO: 5
CDR3 of SEQ ID NO: 6.

The CDRs may be associated with any framework region. Preferably, the framework region is of human origin.

The amino acid variations in the CDR sequences of the isolated antigen binding molecules of the invention may be conservative amino acid substitutions.

In another aspect, there is provided an isolated antigen binding molecule or fragment thereof comprising an antigen binding portion which comprises a variable heavy chain region with at least 80%, 90%, 95%, 98%, 99% or 100% sequence identity to SEQ ID NO: 7 and/or a variable light chain region with at least 80%, 90%, 95%, 98%, 99% or 100% sequence identity to of SEQ ID NO: 8. In an embodiment, the isolated antigen binding molecule or fragment thereof comprises an antigen binding portion which comprises a variable heavy chain region of SEQ ID NO: 7 and a variable light chain region of SEQ ID NO: 8. In an embodiment, the isolated antigen binding molecule or fragment thereof comprises an antigen binding portion in which the variable heavy chain region consists of SEQ ID NO: 7 and the variable light chain region consists of SEQ ID NO: 8.

The isolated antigen binding molecules or fragments thereof of the invention may be capable of specifically binding human G6b-B on platelets and/or on megakaryocytes and/or on disease causing haematopoietic progenitors cells in MPN.

An isolated antigen binding molecule or fragment thereof of the invention may bind to G6b-B with an affinity (K_{D}) of between about 1.0×10⁻¹⁰ M and 3.1×10⁻¹⁰ M. The G6b-B may be in monomeric or dimeric form.

In an embodiment, the isolated antigen binding molecule or fragment thereof can bind to monomeric G6b-B with an affinity (K_{D}) of between about 1.0×10⁻¹⁰ M to about 1.5×10⁻¹⁰ M. In an embodiment, the isolated antigen binding molecule or fragment thereof can bind to monomeric G6b-B with an affinity (K_{D}) of between about 1.2×10⁻¹⁰ M to about 1.4×10⁻¹⁰ M. In an embodiment, the isolated antigen binding molecule or fragment thereof can bind to monomeric G6b-B with an affinity (K_{D}) of about 1.26×10⁻¹⁰ M. In an embodiment, the isolated antigen binding molecule or fragment thereof binds to monomeric G6b-B with an affinity (K_{D}) of 1.26×10⁻¹⁰ M.

In an embodiment, the isolated antigen binding molecule or fragment thereof can bind to dimeric G6b-B with an affinity (K_{D}) of between about 2.0×10⁻¹⁰ M to about 3.1×10⁻¹⁰ M. In an embodiment, the isolated antigen binding molecule or fragment thereof can bind to dimeric G6b-B with an affinity (K_{D}) of between about 2.3×10⁻¹⁰ M to about 2.8×10⁻¹⁰ M. In an embodiment, the isolated antigen binding molecule or fragment thereof can bind to dimeric G6b-B with an affinity (K_{D}) of about 2.6×10⁻¹⁰ M. In an embodiment, the isolated antigen binding molecule or fragment thereof binds to dimeric G6b-B with an affinity (K_{D}) of 2.62×10⁻¹⁰ M.

In another aspect, the invention provides a nucleic acid encoding an antigen binding molecule or a fragment thereof of the invention. In an embodiment, the nucleic acid comprises the heavy chain region defined by SEQ ID NO: 9 and/or the light chain region defined by SEQ ID NO: 10.

In another aspect, the invention provides a vector comprising a nucleic acid of the invention. Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g., ‘phage, or phagemid, as appropriate. For further details see, for example, (Sambrook, J., E. F. Fritsch, and T. Maniatis. (1989), Molecular cloning: a laboratory manual, 2nd ed. Cold Spring Harbor Laboratory, Cold Spring Harbor, New York). Many known techniques and protocols for manipulation of nucleic acid, for example in preparation of nucleic acid constructs, mutagenesis, sequencing, introduction of DNA into cells and gene expression, and analysis of proteins, are described in detail in (Ausubel et al., Current protocols in molecular biology. New York: Greene Publishing Association; Wiley-Interscience, 1992). In an embodiment, the vector is an expression vector. In an embodiment, the vector or expression vector is a plasmid. In an embodiment, the vector comprises nucleic acid encoding a heavy chain region defined by SEQ ID NO: 9 and/or the light chain region defined by SEQ ID NO: 10.

A nucleic acid molecule or vector encoding an antigen binding molecule or fragment thereof of the invention may be expressed using any suitable expression system, for example in a suitable host cell or in a cell-free system.

In another aspect, the invention provides a host cell comprising a vector of the invention. In an embodiment, the vector comprises nucleic acid encoding an antigen binding molecule of the invention. A host cell may be selected from bacterial host cells (prokaryotic systems) such as E. Coli, or eukaryotic cells such as those of yeasts, fungi, insect cells or mammalian cells. Preferably a host cell of the invention is capable of producing the antigen binding molecule or fragment thereof of the invention. The produced antigen binding molecule or fragment thereof may be enriched by means of selection and/or isolation.

In an embodiment, the host cell is a mammalian cell. In an embodiment, the mammalian cell is HEK 293 cell or a derivative thereof, for example a HEK293 6E cell.

An antigen binding molecule or fragment thereof of the invention may also be produced by chemical synthesis. The obtained antigen binding molecule may be enriched by means of selection and/or isolation.

According to a further aspect, the invention provides a pharmaceutical composition that comprises an isolated antigen binding molecule or fragment thereof, nucleic acid, vector and/or host cell of the invention, optionally together with one or more pharmaceutically acceptable excipients or diluents.

Isolated antigen binding molecules or fragments thereof, nucleic acids, vectors or host cells of the invention can be formulated into pharmaceutical compositions using established methods of preparation (Gennaro, A. L. and Gennaro, A. R. (2000) Remington: The Science and Practice of Pharmacy, 20th Ed., Lippincott Williams & Wilkins, Philadelphia, Pa.). To prepare the pharmaceutical compositions, pharmaceutically inert inorganic or organic excipients can be used. To prepare for example pills, powders, gelatin capsules or suppositories, lactose, talc, stearic acid and its salts, fats, waxes, solid or liquid polyols, natural and hardened oils are examples of pharmaceutically acceptable excipients which can be used. Suitable excipients for the production of solutions, suspensions, emulsions, aerosol mixtures or powders for reconstitution into solutions or aerosol mixtures prior to use include water, alcohols, glycerol, polyols, and suitable mixtures thereof as well as vegetable oils.

A pharmaceutical composition of the invention may be administered via any parenteral or non-parenteral (enteral) route that is therapeutically effective. Parenteral application methods include, for example, intracutaneous, subcutaneous, intramuscular, intratracheal, intranasal, intravitreal or intravenous injection and infusion techniques, e.g. in the form of injection solutions, infusion solutions or mixtures, as well as aerosol installation and inhalation, e.g. in the form of aerosol mixtures, sprays or powders. A pharmaceutical composition of the invention can be administered systemically or topically in formulations containing conventional non-toxic pharmaceutically acceptable excipients or carriers, additives and vehicles as desired. A combination of intravenous and subcutaneous infusion and/or injection might be most convenient in case of compounds with a relatively short serum half-life. Preferably, the pharmaceutical composition is administered intravenously. The pharmaceutical composition may be an aqueous solution, an oil-in water emulsion or a water-in-oil emulsion.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using for example, isotonic vehicles such as Sodium Chloride Injection, Ringer's Injection, Lactated Ringer's Injection. Preservatives, stabilisers, buffers, antioxidants and/or other additives may be included, as required.

The compositions are preferably administered to an individual in a “therapeutically effective amount”, this being sufficient to show benefit to the individual. The optimal dosage will depend on the biodistribution of the isolated antigen binding molecule or fragment thereof, the mode of administration, the severity of the disease/disorder being treated as well as the medical condition of the patient. If wanted, the isolated antigen binding molecule or fragment thereof may be given in a sustained release formulation, for example liposomal dispersions or hydrogel-based polymer microspheres, like PolyActive™ or OctoDEX™ (cf. Bos et al., Business Briefing: Pharmatech 2003: 1-6). Other sustained release formulations available are for example PLGA based polymers (PR pharmaceuticals), PLA-PEG based hydrogels (Medincell) and PEA based polymers (Medivas). Prescription of treatment, e.g., decisions on dosage etc, is within the responsibility of general practitioners and other medical doctors, and typically takes account of the disorder to be treated, the condition of the individual patient, the site of delivery, the method of administration and other factors known to practitioners.

The pharmaceutical composition may also contain additives, such as, for example, fillers, binders, wetting agents, glidants, stabilizers, preservatives, emulsifiers, and furthermore solvents or solubilizers or agents for achieving a depot effect. The latter is that fusion proteins may be incorporated into slow or sustained release or targeted delivery systems, such as liposomes and microcapsules.

An isolated antigen binding molecule of fragment thereof, nucleic acid, vector, host cell or composition of the invention may be suitable for and may be used in the treatment or prevention of a disorder or disease.

In an aspect, the invention provides an isolated antigen binding molecule or fragment thereof, nucleic acid, vector, host cell, or composition of the invention may be used in a method of treatment of the human or animal body, such as a method of treatment of diseases or disorders associated with dysregulated platelet homeostasis, such as for example in myeloproliferative or thrombotic diseases and disorders in a patient, said method comprising administering to said patient an effective amount of the isolated antigen binding molecule or fragment thereof, nucleic acid, expression vector, host cell, or composition of the invention. In a preferred embodiment, the patient is a human. In an embodiment, diseases or disorders may comprise one or more of a myeloproliferative neoplasm, thrombocythemia, myelofibrosis, thrombocytosis, thrombocytopenia (for example macrothrombocytopenia, microthrombocytopenia and normothrombocytopenia), haemophilia, Bernard-Soulier syndrome, Glanzmann thrombasthenia, alpha granule deficiency, delta storage pool deficiency, Scott syndrome and myelodysplastic syndromes.

In another aspect, the invention also provides an isolated antigen binding molecule or fragment thereof, nucleic acid, vector, host cell, or composition for use in medicine, as well as the use of an isolated antigen binding molecule or fragment thereof, nucleic acid, expression vector, host cell, or composition of the present invention in the manufacture of a medicament for the treatment of diseases or disorders associated with dysregulated platelet homeostasis, such as for example in myeloproliferative or thrombotic diseases and disorders. In an embodiment, diseases or disorders may comprise one or more of a myeloproliferative neoplasm, thrombocythemia, myelofibrosis, thrombocytosis, thrombocytopenia (for example macrothrombocytopenia, microthrombocytopenia and normothrombocytopenia), haemophilia, Bernard-Soulier syndrome, Glanzmann thrombasthenia, alpha granule deficiency, delta storage pool deficiency, Scott syndrome and myelodysplastic syndromes.

In yet a further aspect, the invention provides an isolated antigen binding molecule or fragment thereof, nucleic acid, vector, host cell, or composition of the invention for use in treating diseases or disorders associated with dysregulated platelet homeostasis, such as for example in myeloproliferative or thrombotic diseases and disorders. In an embodiment, diseases or disorders may comprise one or more of a myeloproliferative neoplasm, thrombocythemia, myelofibrosis, thrombocytosis, thrombocytopenia (for example macrothrombocytopenia, microthrombocytopenia and normothrombocytopenia), haemophilia, Bernard-Soulier syndrome, Glanzmann thrombasthenia, alpha granule deficiency, delta storage pool deficiency, Scott syndrome and myelodysplastic syndromes.

Furthermore, an isolated antigen binding molecule or fragment thereof, nucleic acid, vector, host cell or composition of the present invention may be administered alone or in combination with other therapeutic agents, either simultaneously, sequentially or separately, dependent upon the condition to be treated.

In this context, the word “homeostasis” as it relates to platelets may refer to the production of platelets and therefore the number of platelets present in a sample or a patient. The word “homeostasis” as it relates to platelets may also refer to the reactivity of platelets, including but not limited to, properties relating to their activation, adhesion, coagulation activity, and vaso-modulating activity. The word “homeostasis” as it relates to platelets may also refer to the size of platelets, for example large or small platelets as compared to a normal sized platelet, as the skilled person would understand.

The present invention further provides an isolated antigen binding molecule or fragment thereof, nucleic acid, vector, host cell, or composition of the invention, which is suitable to be administered with another therapeutic. In an embodiment, the another therapeutic may be one or more of heparin or a derivative thereof, radiotherapy, or a chemotherapeutic agent.

The further therapeutic agent may be administered simultaneously, separately or sequentially when used with an isolated antigen binding molecule or fragment thereof, nucleic acid, vector, host cell, or composition of the invention.

Further therapeutic agents described herein may operate synergistically with the isolated antigen binding molecule or fragment thereof, nucleic acid, vector, host cell, or composition of the present invention.

Whilst not wishing to be bound by theory, the ability of the isolated antigen binding molecule or fragment thereof, nucleic acid, vector, host cell, or composition of the invention to synergise with a further therapeutic agent to enhance treatment of the indicated diseases may not be due to immune effector mechanisms but rather may be a direct consequence of the antigen binding molecule binding to G6b-B at the epitope at residues on human G6b-B equivalent to Asp 24, Arg 26 and Gly 124 of FIG. 14, residues equivalent to Pro19 to Arg26 and His121 to Gly124 of FIG. 14, or residues equivalent to Arg26, Asp 24, Gly25, His121, Val122, Leu123, Gly124, Asp125, Ser22, Asp29, Leu23, Val131, Gly20, Asp32, Ala21 and Pro19 of FIG. 14.

In yet a further aspect, the invention provides a method of modulating platelet homeostasis in a subject, comprising administering to the subject an effective amount of isolated antigen binding molecule or fragment thereof, nucleic acid, vector, host cell, or composition of the invention.

The term “antigen binding molecule” generally refers to a proteinaceous binding molecule that is based on an immunoglobulin. Such antigen binding molecules are generally encoded by amino acids to form a polypeptide. Typical examples of an antigen binding molecule are derivatives or functional fragments of an immunoglobulin which retain the binding specificity. Techniques for the production of antigen binding molecules including antibodies and fragments thereof are well known in the art. The terms “antigen binding molecule” and “antibody” also include immunoglobulins (Ig's) of different classes (i.e. IgA, IgG, IgM, IgD and IgE) and subclasses (such as IgG1, IgG2 etc.). Illustrative examples of an antigen binding molecules or fragments thereof include Fab fragments, F(ab′)2, Fv fragments, single-chain Fv fragments (scFv), diabodies, domain antibodies or bispecific antibodies (Holt L J et al., Trends Biotechnol. 21(11), 2003, 484-490). Examples also include a dAB fragment which consists of a single CH domain or VL domain which alone is capable of binding an antigen. The definition of the term “antibody” thus also includes embodiments such as chimeric, single chain and humanized antibodies. Antibodies may be monoclonal (mAb) or polyclonal.

The isolated antigen binding molecule or fragment thereof may be an antibody-like molecule which includes the use of CDRs separately or in combination in synthetic molecules such as SMIPs and small antibody mimetics.

The invention also includes within its scope isolated antigen binding molecules or fragments thereof comprising the amino acid sequence as set out in SEQ ID NOs: 1-8, polynucleotides comprising the nucleic acid sequences as set out in SEQ ID NOs: 9-10 and sequences having substantial identity thereto, for example, 70%, 80%, 85%, 90%, 95%, 99% or 100% identity thereto.

The percent identity of two amino acid sequences or of two nucleic acid sequences is generally determined by aligning the sequences for optimal comparison purposes (e.g., gaps can be introduced in the first sequence for best alignment with the second sequence) and comparing the amino acid residues or nucleotides at corresponding positions. The “best alignment” is an alignment of two sequences that results in the highest percent identity. The percent identity is determined by comparing the number of identical amino acid residues or nucleotides within the sequences (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul, 1990, PNAS, 87(6):2264-8, modified as in Karlin and Altschul, 1993, PNAS, 90(12):5873-5877 The NBLAST and XBLAST programs of Altschul et al., 1990, J. Mol. Biol., 215:403-10 have incorporated such an algorithm. BLAST nucleotide searches can be performed with the NBLAST program, score=100, word length=12 to obtain nucleotide sequences homologous to a nucleic acid molecules of the invention. BLAST protein searches can be performed with the XBLAST program, score=50, word length=3 to obtain amino acid sequences homologous to a protein molecules of the invention. To obtain gapped alignments for comparison purposes, Gapped BLAST can be utilized as described in Altschul et al. (1997). Alternatively, PSI-Blast can be used to perform an iterated search that detects distant relationships between molecules (Id.). When utilizing BLAST, GappedBLAST, and PSI-Blast programs, the default parameters of the respective programs (e.g., XBLAST and NBLAST) can be used. See http://www.ncbi.nlm.nih.gov. Another example of a mathematical algorithm utilized for the comparison of sequences is the algorithm of Myers and Miller. The ALIGN program (version 2.0) which is part of the GCG sequence alignment software package has incorporated such an algorithm. Other algorithms for sequence analysis known in the art include ADVANCE and ADAM as described in Torellis and Robotti (1994); and FASTA described in Pearson and Lipman (1988). Within FASTA, ktup is a control option that sets the sensitivity and speed of the search.

An isolated antigen binding molecule or fragment thereof of the invention may comprise one or more mutated amino acid residues. The terms “mutated”, “mutant” and “mutation” in reference to a nucleic acid or an isolated antigen binding molecule or fragment thereof of the invention refers to the substitution, deletion, or insertion of one or more nucleotides or amino acids, respectively, compared to the “naturally” occurring nucleic acid or polypeptide, i.e. to a reference sequence that can be taken to define the wild-type. For example, the heavy and light chains of the antigen binding molecule or fragment thereof defined by SEQ ID NOs: 7 and 8, as obtained by immunization and as described herein may be taken as a wild-type sequence.

In some embodiments a mutation may be a substitution wherein the substitution is a conservative substitution. Conservative substitutions are generally the following substitutions, listed according to the amino acid to be mutated, each followed by one or more replacement(s) that can be taken to be conservative: Ala→Gly, Ser, Val; Arg→Lys; Asn→Gln, His; Asp→Glu; Cys→Ser; Gln→Asn; Glu→Asp; Gly→Ala; His→Arg, Asn, Gln; Ile→Leu, Val; Leu→Ile, Val; Lys→Arg, Gln, Glu; Met→Leu, Tyr, He; Phe→Met, Leu, Tyr; Ser→Thr; Thr→Ser; Trp→Tyr; Tyr→Trp, Phe; Val→He, Leu. Other substitutions are also permissible and can be determined empirically or in accord with other known conservative or non-conservative substitutions.

It is contemplated that 1, 2 or 3 conservative substitutions may be made in the CDRs of the antigen binding molecules of the invention.

The term “antigen binding portion” is used herein to mean the portion of the antigen binding molecule or fragment thereof that comprises one or more complementarity determining regions (CDRs) and bind antigen in the same way as antibody or antibody like molecule. The antigen binding portion may be based on an scFv fragment. The antigen binding portion may be based on an antibody mimetic.

As used herein, the antibody “17-4” refers to a monoclonal IgG1 antibody comprising a heavy chain of SEQ ID NO: 7 and a light chain of SEQ ID NO: 8.

The term “epitope”, also known as the “antigenic determinant”, refers to the portion of an antigen to which an antigen binding molecule or fragment thereof specifically binds, thereby forming a complex. Thus, the term “epitope” includes any molecule or protein determinant capable of specific binding to an antigen binding molecule or fragment thereof.

The term “specific” in this context, or “specifically binding”, also used as “directed to”, means in accordance with this invention that the antigen binding molecule or fragment thereof is capable of specifically interacting with and/or binding to a specific antigen or ligand or a set of specific antigens or ligands but does not essentially bind to other antigens or ligands. Such binding may be exemplified by the specificity of a “lock-and-key-principle”. Antigen binding molecules or fragments thereof are said to “bind to the same epitope” if the antigen binding molecule cross-compete so that only one antigen binding molecule can bind to the epitope at a given point of time, i.e. one antigen binding molecule prevents the binding or modulating effect of the other.

Binding may be considered specific when the binding affinity is higher than 10⁻⁶ M or 10⁻⁷ M. In particular, binding is considered specific when binding affinity is about 10⁻⁸ to 10⁻¹¹ M (KD), or of about 10⁻⁹ to 10⁻¹¹ M or even higher. If necessary, nonspecific binding of a binding site can be reduced without substantially affecting specific binding by varying the binding conditions.

The term “isolated” as applied to an antigen binding molecule or fragment thereof and as used herein refers to an antigen binding molecule or fragment thereof that has been identified and separated and/or recovered from a component of its natural environment. Contaminant components of its natural environment are matter that would interfere with diagnostic or therapeutic uses for the antigen binding molecule or fragment thereof, and may include enzymes, hormones, and other proteinaceous or non-proteinaceous solutes. In some embodiments the antigen binding molecule or fragment thereof is purified to greater than 95% by weight of antigen binding molecule or fragment thereof as determined by the Lowry method, such as more than 99% by weight. In some embodiments the antigen binding molecule or fragment thereof is purified to homogeneity as judged by SDS-PAGE under reducing or non-reducing conditions using Coomassie blue or, preferably, silver stain. An isolated antigen binding molecule or fragment thereof may in some embodiments be present within recombinant cells with one or more component(s) of the antigen binding molecule or fragment thereof's natural environment not being present. Typically an isolated antigen binding molecule is prepared by at least one purification step.

An isolated antigen binding molecule or fragment thereof of the invention as described herein may be used in any suitable recombinant format, for example as an Fv fragment, a scFv, a univalent antibody lacking a hinge region, a minibody, a Fab fragment, a Fab′ fragment, a F(ab′)2 fragment. A recombinant antigen binding molecule or fragment thereof of the invention may also comprise constant domains (regions) such a human IgG constant region, a CH1 domain (as Fab fragments do) and/or an entire Fc region. Alternatively, an isolated antigen binding molecule or fragment thereof of the invention may also be a full length (whole) antibody. Alternatively, an antigen binding molecule or fragment thereof of the invention may be a bispecific antibody.

An isolated antigen binding molecule or fragment thereof of the invention is preferably an antibody capable of binding to human G6b-B. The term G6b-B includes variants, isoforms and species homologs of human G6b-B. G6b-B is also designated Megakaryocyte and platelet inhibitory receptor G6b. Human G6b-B has the UniProt accession number 095866 and a sequence as defined in FIG. 14. Accordingly, antigen binding molecules or fragments thereof of the invention may, in certain cases, cross-react with G6b-B from species other than human, or other proteins which are structurally related to human G6b-B (e.g. human G6b-B homologs).

Isolated antigen binding molecules or fragments thereof that bind to the same epitope as an isolated antigen binding molecule or fragment thereof described herein are within the scope of the invention. Also within the scope of the invention are isolated antigen binding molecules or fragments thereof that compete with an antigen binding molecule or fragment thereof of the invention for binding to G6b-B, e.g. to competitively inhibit binding of an isolated antigen binding molecule or fragment thereof of the invention to G6b. To determine competitive inhibition, a variety of assays known to one of ordinary skill in the art can be employed. For example, cross-competition assays can be used to determine if an isolated antigen binding molecule or fragment thereof competitively inhibits binding to G6b-B by another antigen binding molecule or fragment thereof. These include cell-based methods employing flow cytometry or solid phase binding analysis.

A “bispecific” or “bifunctional” antibody molecule is an antigen binding molecule that has two different epitope/antigen binding sites, and accordingly has binding specificities for two different target epitopes. These two epitopes may be epitopes of the same antigen or of different antigens. In contrast thereto a “bivalent antibody” may have binding sites of identical antigenic specificity. In an embodiment, an antigen binding molecule of the invention may be a bispecific antibody. The bispecific antibody may comprise (a) a variable region comprising a heavy chain variable region and a light chain variable region as defined above, wherein said variable region comprises a first binding site capable of binding to human G6b-B, for example at the epitope comprising at least one or more, two or more or three of residues equivalent to Asp 24, Arg 26 and Gly 124 of FIG. 14 or at least one or more, two or more, three or more of residues equivalent to Pro19 to Arg26 and His121 to Gly124 of FIG. 14 or at least one or more, two or more, three or more of residues equivalent to Arg26, Asp 24, Gly25, His121, Val122, Leu123, Gly124, Asp125, Ser22, Asp29, Leu23, Val131, Gly20, Asp32, Ala21 and Pro19 of FIG. 14 and (b) a heavy chain variable region and a light chain variable region of an antigen binding molecule comprising a second binding site other than G6b-B. In an embodiment, the second binding site may bind to CD34, Mpl, GPVI, CLEC-2, Fc gammaRIIA, integrin alpha or interleukin beta3.

Methods of making an isolated antigen binding molecule or fragment thereof, such as an antibody, are well known in the art. The skilled person may use hybridoma technology for example, or may use recombinant DNA technology to clone the respective antibody sequence into a vector, such as an expression vector. Methods of making a bispecific antibody molecule are known in the art, e.g. recombinant DNA technology, chemical conjugation of two different monoclonal antibodies or for example, also chemical conjugation of two antibody fragments, for example, of two Fab fragments. Alternatively, bispecific antibody molecules are made by quadroma technology, which is by fusion of the hybridomas producing the parental antibodies. Because of the random assortment of H and L chains, a potential mixture of ten different antibody structures are produced of which only one has the desired binding specificity. A bispecific antibody molecule of the invention can act as a monoclonal antibody (mAb) with respect to each target. In some embodiments the antibody is chimeric, humanized or fully human. A bispecific antibody molecule may for example be a bispecific tandem single chain Fv, a bispecific Fab2, or a bispecific diabody.

All of the features disclosed in this specification may be combined in any combination, including with any aspect or any embodiment. Each feature disclosed in this specification may be replaced by an alternative feature serving the same, equivalent, or similar purpose. Thus, unless expressly stated otherwise, each feature disclosed is only an example of a generic series of equivalent or similar features.

### BRIEF DESCRIPTION OF THE DRAWINGS

FIG. 1—(a,b) shows the sequences of the CDRs in the heavy and light chain of antigen binding molecules of the invention. (c-f) Amino acid and nucleotide sequences of the heavy and light chain of antigen binding molecules of the invention.

FIG. 2—illustrates that the anti-G6b mouse monoclonal antibody 17-4 produces a severe and sustained thrombocytopenia in G6b-humanised mice. 0.45 μg/g bodyweight of 17-4 or phosphate buffered saline (PBS) was injected into G6b-humanized mice (2 per group) and platelet count and volume in the blood was measured at the indicated time points.

FIG. 3—illustrates that the anti-G6b monoclonal antibody 17-4 increases heparin-mediated potentiation of human washed platelet aggregation. Human washed platelets were pre-incubated for 1.5 minutes with (1) 3 μg/ml anti-FcγRIIA blocking (IV.3) F(ab′)2; (2) 5 μg/ml anti-G6b monoclonal antibody (mAb) 17-4 or IgG; (3) indicated concentration of unfractionated heparin or HEPES buffered saline (HBS) before activation with 0.3 μg/ml collagen. (A) mean aggregation traces and (B) area under the curve (AUC) analysis. Mean±SEM, n=4-5. One-way ANOVA: * P<0.05; ** P<0.005; *** P<0.001.

FIG. 4—shows a dose-dependent effect of monoclonal antibody 17-4 on heparin potentiation of human washed platelet aggregation. Human washed platelets were pre-incubated for 1.5 minutes with (1) 3 μg/mlanti-FcγRIIA blocking (IV.3) F(ab′)2; (2) indicated concentration of anti-G6b monoclonal antibody (mAb) 17-4 or PBS; (3) 10 μg/ml unfractionated heparin or HEPES buffered saline (HBS) before activation with 0.3 μg/ml collagen. (A) mean aggregation traces and (B) area under the curve (AUC) analysis. Mean±SEM, n=2.

FIG. 5—demonstrates that the 17-4 Fab fragment potentiates the effect of heparin on human washed platelets. Human washed platelets were pre-incubated for 1.5 minutes with (1) 5 μg/ml anti-G6b antibody 17-4 Fab fragments or PBS and (2) 10 μg/ml unfractionated heparin or HEPES buffered saline (HBS) before activation with 0.3 μg/ml collagen. (A) mean aggregation traces and (B) area under the curve (AUC) analysis. Mean±SEM, n=3. One-way ANOVA: * P<0.05.

FIG. 6—illustrates that the monoclonal antibody 17-4 increases heparin potentiation of humanised G6b mouse washed platelet aggregation. Humanised G6b mouse washed platelets were pre-incubated for 1.5 minutes with (1) 5 μg/ml anti-G6b monoclonal antibody (mAb) 17-4 or PBS and (2) indicated concentration of unfractionated heparin or HEPES buffered saline (HBS) before activation with 0.3 μg/ml collagen. (A) Mean aggregation traces and (B) area under the curve (AUC) analysis. Mean±SEM, n=2-6. One-way ANOVA: ** P<0.005; *** P<0.001

FIG. 7—uses flow cytometry to demonstrate strong binding of 17-4 to human platelets. Human platelets were stained with indicated anti-G6b monoclonal antibodies or IgG control at the indicated concentration, fixed and subsequently stained with anti-mouse Alexa488-conjugated antibody. Mean fluorescence intensity was measured by flow cytometry.

FIG. 8—illustrates that the 17-4 antibody robustly binds to recombinant monomeric and dimeric human G6b-B, but not to mouse G6b-B in an in vitro binding assay. Wells of 96-well plate were coated with the indicated proteins overnight. Following blocking, wells were incubated with the indicated anti-G6b monoclonal antibodies (10 μg/ml) or IgG control. Antibody binding was detected with HRP-conjugated anti-mouse antibody and 3,3,5,5-tetramethylbenzidine (TMB) substrate; absorbance was measured with a plate reader at 450 nm.

FIG. 9—shows immunofluorescent staining of G6b-B in spread platelets with 17-4. Human platelets were spread on a collagen I coated surface for 45 min; platelets were fixed and, when indicated, permeabilized and stained with the anti-G6b mouse monoclonal antibody 17-4 (5 μg/ml) followed by anti-mouse Alexa488-conjugated antibody; Images were acquired by confocal microscopy; scale bar: 20 μm.

FIG. 10—uses immunohistochemistry to show robust and specific binding of 17-4 on megakaryocytes in the bone marrow. Serial histologic sections of a bone marrow biopsies from (A) a normal control individual and (B,C) G6b-B deficient individuals, stained with the anti-G6b monoclonal antibody 17-4. Staining for G6b-B is entirely negative in the megakaryocytes and platelets from G6b-B deficient individuals. Figure modified from: Hofmann, Geer et al., Blood, 2018.

FIG. 11—is a ribbon representation of the refined X-ray crystal structure of the G6b-B ECD:DP12:Fab complex. The two chains of the G6b-B dimer (pink and grey) are in the centre of the figure with DP12 bound at their dimer interface (blue sticks). A single anti-G6b Fab molecules (VH:VL chains coloured green:blue and magenta:yellow respectively) are shown bound to each G6b-B ECD domain.

FIG. 12—is a ribbon representation of the binding interface for one of the G6b-B ECD:Fab complexes in the crystal asymmetric unit. G6b-B ECD (grey), Fab VH domain (magenta) and Fab VL domain (yellow).

FIG. 13—illustrates G6b-B ECD:Fab residue interactions between G6b-B and exemplary antibody 17-4.

A: G6b-B ECD:Fab residue interactions between G6b-B chain E and Fab chains A and B. B: G6b-B ECD:Fab residue interactions between G6b-B chain F and Fab chains C and D

Legend to the figure: ‡ The type of contact: H-bond (H), Ionic (I), or Distance (D); † The distance (in Angstroms) between the centroids of the interacting atoms. When multiple interactions are aggregated into a single entry, this value is the average distance. § Denotes whether any of the interacting atoms in the entry are backbone (b) or not (−). The first character represents the Fab residue and the second the G6b-B residue.

FIG. 14—is the sequence of human G6b-B (Uniprot accession 095866).

### METHODS AND MATERIALS

### Sequencing of Exemplary Antibody 17-4 (Mouse IgG1)

Total RNA was isolated from hybridoma cells following the technical manual of TRIzol® Reagent. Total RNA was then reverse-transcribed into cDNA using either isotype-specific anti-sense primers or universal primers following the technical manual of PrimeScript™ 1st Strand cDNA Synthesis Kit. Antibody fragments of VH, VL, CH and CL were amplified according to the standard operating procedure (SOP) of rapid amplification of cDNA ends (RACE) of GenScript. Amplified antibody fragments were cloned into a standard cloning vector separately. Colony PCR was performed to screen for clones with inserts of correct sizes. No less than five colonies with inserts of correct sizes were sequenced for each fragment. The sequences of different clones were aligned and the consensus sequence was provided.

### Production of Recombinant Anti-G6b-B Fab Fragment

DNA constructs of 17-4 Fab light and heavy chains were synthesised and inserted into the pTT5 mammalian expression vector. HEK293 6E mammalian cells, grown at a density of 1.8-2×10⁶ cells/ml in F17 media plus 0.1% v/v F68 pluronic and 8 mM glutamine, were transiently transfected with both constructs using 2.8 μg/mL PEI (DNA concentration 0.375 μg/ml for each construct). The cells were fed 24 hr post transfection with HYPEP 1510 (0.32% w/v final conc) and cultured to co-express human 17-4 Fab. 6 days post-transfection the supernatant containing the expressed protein was harvested by centrifugation and stored at 4° C., prior to purification. Recombinant Fab fragment was isolated using IMAC (Nickel Sepharose excel, GE Healthcare LifeSciences), and further purified on a 120 ml HiLoad 16/60 Superdex 75 column (GE Healthcare LifeSciences). The size exclusion column was equilibrated and run in PBS.

### Production of Recombinant G6b-B

G6b-B ECD (18-133, [N32D, S67A, S68A, S69A, T71A]) was expressed following transient transfection in Hek293 6E cells. Conditioned media containing expressed protein was harvested by centrifugation after 7 days. Media was diluted 1:2 with 20 mM sodium phosphate, pH 7.0, and expressed protein purified by cation exchange chromatography through a 5 ml HiTrap SP column (GE Healthcare LifeSciences). Bound proteins were eluted with a stepwise gradient elution of 0 to 1 M NaCl in 20 mM sodium phosphate pH 7.0, over 20 column volumes. Fractions were analysed by SDS PAGE under non-reducing conditions prior to concentration using 3 kDa MWCO centrifugal concentration units (Sartorius), followed by further purification on a 120 ml HiLoad 16/60 Superdex 75 column (GE Healthcare LifeSciences). The size exclusion column was equilibrated and run in 20 mM Hepes, pH 7.1, 150 mM NaCl.

### Production of Recombinant G6b-B-Fab Complex

Pure G6b-B ECD fractions were identified by non-reducing SDS PAGE prior to incubation with recombinant anti-G6b-B Fab domain to allow G6b-B-Fab complex formation. Incubations were carried out at a 1.5 molar excess of G6b-B for 2 hrs at room temperature. G6b-B-Fab complex was then purified from unbound protein by size exclusion chromatography on a 12 0 ml HiLoad 16/60 Superdex 75 column (GE Healthcare LifeSciences), using the same buffer as above. Fractions containing pure G6b-Fab complex were identified by SDS PAGE, and adjusted to 75 mM NaCl prior to concentration using 50 kDa MWCO centrifugal concentration units (Sartorius). Purified G6b-B-Fab complex was supplied for crystallisation at 12.05 mg/ml in buffer solution of 20 mM Hepes pH 7.1, 75 mM NaCl. All purified proteins and complexes were characterised by mass spectrometry and SDS PAGE.

### Crystallography

Crystals of the G6b-B ECD (aa18-133, N32D, S67A, S68A, S69A, T71A):anti-G6b-B Fab were obtained by sitting-drop vapour diffusion. The preformed G6b-B:Fab complex at 12 mg/ml, in a buffer solution of 20 mM HEPES, 75 mM NaCl, pH 7.1 was incubated for 1 hour at 4° C. with 2 mM DP12 (Iduron Cat. No. HO12). This protein sample was then microfuged at 16,000 g, for 10 minutes at 4° C., before drops were set up using 150 nl sample and 100 nl reservoir solution. Reservoir conditions were 50 mM MES pH6.2, 10% PEG 550MME, 5% glycerol, and 50 mM CaCl₂. Crystallisation plates were incubated at 20° C. and flat plate crystals appeared within 3 days. The crystal was harvested straight out of the growth drop into liquid nitrogen. Diffraction data were collected at 100K on beamline 103 at Diamond Light Source and processed by XDS1 and Aimless2 via AutoPROC3. The crystal had the space group C2 with the cell dimensions of a=183.80, b=72.34, c=131.04, alpha=90.0°, beta=124.52°, gamma=90.0°, and gave a 97% complete dataset to 3.1 Å. Data processing statistics are summarised in Table 1. Calculation of the Mathews coefficient (CCP4 Suite4) suggested that there was sufficient volume in the asymmetric unit for two G6b-B ECD:Fab complexes.

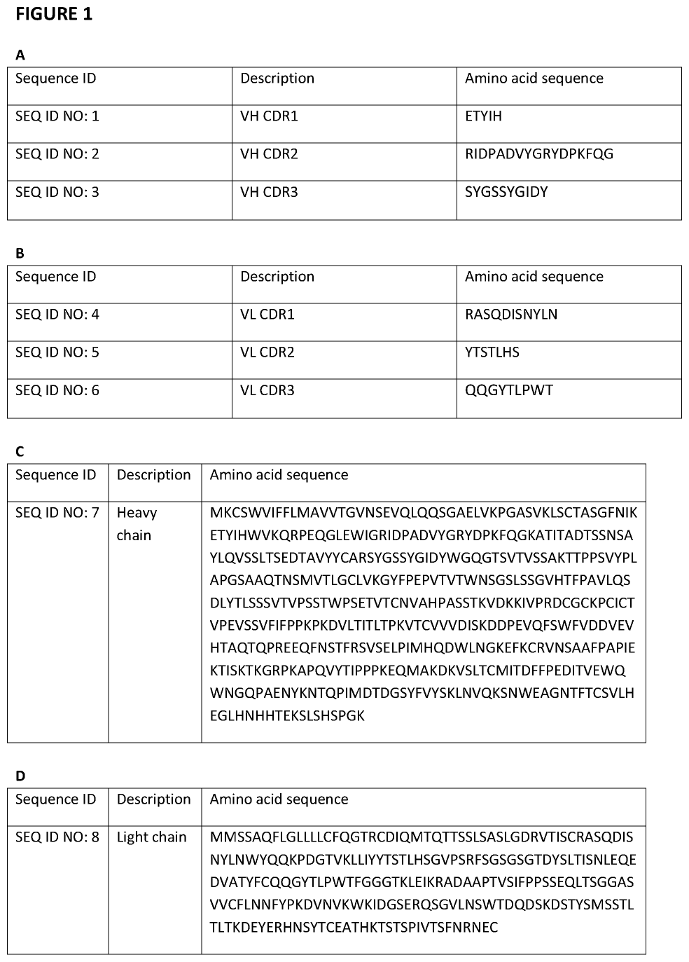

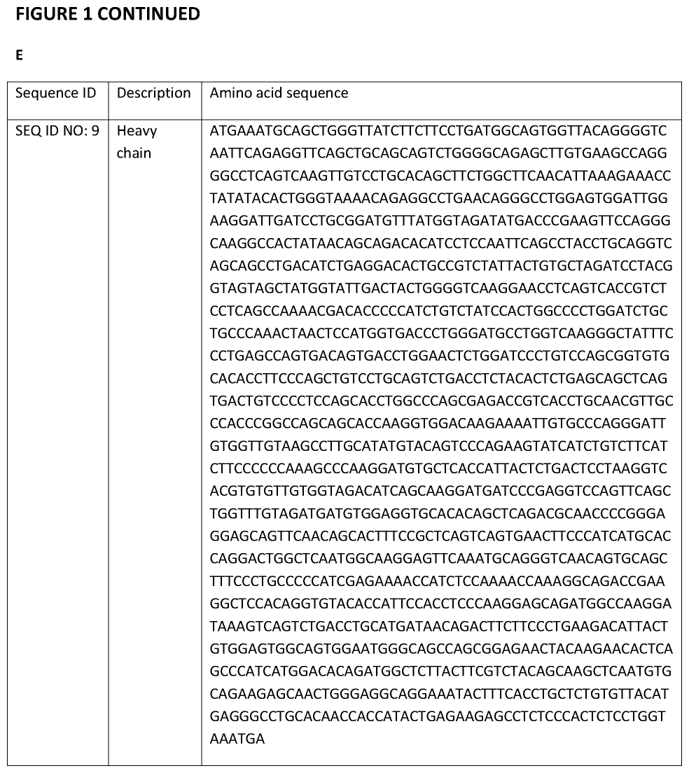

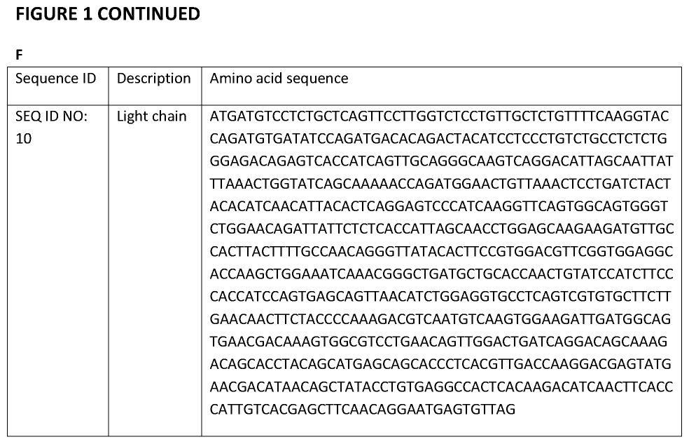

The structure was initially solved by molecular replacement using the program Phaser5 and with a model of the Fab generated from the PDB structure 4K2U as the search model. This resulted in the placement of two Fab molecules in the asymmetric unit. Examination of the resulting electron density maps showed substantial unmodeled density in the vicinity of the CDR regions of both Fab molecules which was assumed to be bound G6b-B ECD. Multiple rounds of model building in Coot6 and refinement using Refmac57 resulted in the most of the of G6b-B ECD chain being built. Residual density at that stage was identified as a single molecule of DP12 bound in a highly positively charged groove formed at the interface of two dimerised G6b-B molecules although only eight of the twelve saccharide units were able to be fully resolved. The final model therefore represents a complex of G6b-B ECD, DP12 and Fab chains in the ratio 2:1:2 respectively (FIG. 1). The refined structure of G6b-B ECD chain has observable electron density for residues Pro19 to Thr38, Arg43 to Arg83 and Ile91 to Cys129. The G6b-B ECD as expected is shown to be a member of the IgV superfamily with the solved structure comprises two antiparallel β-sheets formed by strands ABDE and A′CC′FG. There is also clear electron density for 0-linked glycosylation at Thr71 in both copies of G6b-B ECD. Final refinement statistics for the G6b-B-ECD:DP12:Fab dimer complex are given in Table 2.

### Human Washed Platelets

Human blood was collected from healthy, drug free volunteers into 1:10 (v:v) sodium citrate. Further anticoagulation was provided by 1:9 (v:v) acid citrate dextrose (97 mM sodium citrate, 111 mM glucose and 71 mM citric acid) before centrifugation at 200×g, 20 minutes, RT to separate PRP and red blood cells. PRP was retained and centrifuged at 1000×g, 10 minutes, RT, in the presence of 10 μg/ml PGI₂, to pellet platelets. Platelets were resuspended in modified Tyrodes-HEPES buffer (134 mM NaCl, 2.9 mM KCl, 0.34 mM Na₂HPO₄, 12 mM NaHCO₃, 20 mM HEPES, 1 mM MgCl₂ and 5 mM glucose, pH7.3) and 1:8 (v:v) acid-citrate-dextrose. Platelets were pelleted again as above and resuspended in Tyrodes-HEPES buffer and counted using a Coulter Z2 Particle Count and Size Analyzer (Beckman Coulter Ltd). Platelets were diluted to 2×10⁸/ml for aggregation experiments.

### Mouse Washed Platelets

Mice were terminally anaesthetised with isoflurane and asphyxiated with CO₂. Blood was collected from the vena cava using a 25 gauge needle and a 1 ml syringe containing 200 μl acid citrate-dextrose solution. Following collection, blood was immediately diluted in 200 μl of Tyrodes-HEPES buffer. Whole blood was spun in a microcentrifuge at 2000 rpm, 5 minutes, RT and PRP with the top third of erythrocytes retained. PRP was separated from erythrocytes by centrifugation at 200×g, 6 minutes, RT in a swinging bucket centrifuge and collected into a fresh tube. Tyrodes-HEPES buffer was added to the PRP to give a total volume of 1 ml and platelets pelleted by centrifugation at 1000×g, 6 minutes, RT in the presence of 10 μg/ml PGI₂. Platelets were resuspended in Tyrodes-HEPES buffer before diluting to 2×10⁸/ml for aggregation experiments.

### Aggregometry

Washed platelets (2×10⁸/ml) or PRP were incubated at 37° C. for 2 minutes, followed by a further 1 minutes under stirring conditions (1,200 rotations per minute) in a Chrono-log 700 lumi-aggregometer (Havertown). Agonists (1:100 dilution) were either added directly following this incubation and measurements started, or antibody/inhibitor treatments started. Unless stated otherwise, antibodies and inhibitors were incubated for 1.5 minutes. Area under the curve (AUC) was calculated by the AGGRO/LINK8 software (Chronolog) for 6 minutes following addition of agonist. Averaged traces presented were generated using Microsoft Excel (Redmond), by exporting data points of traces from AGGRO/LINK8.

### Effects of Antibodies In Vivo

0.45 μg/g bodyweight of 17-4 or PBS was injected intravenously into G6b-humanized mice. At the indicated time points, blood was drawn from a superficial vein or at the end of the experiment, from mice terminally anaesthetised with isoflurane and asphyxiated with CO₂ as described above. Platelet count and volume in the blood were measured using an ABX Pentra 60 hematological counter (Horiba Medical).

### Flow Cytometry

Human whole blood was collected, diluted and incubated with indicated anti-G6b monoclonal antibodies or IgG control at the indicated concentration for 30 minutes at RT. Samples were fixed with 1% ice cold formalin, centrifuged (1000×g, 5 min, RT), washed once in PBS, and subsequently stained with anti-mouse Alexa488-conjugated antibody (Invitrogen) for 30 min at RT. Mean fluorescence intensity was measured using an Accuri C6 flow cytometer (BD Biosciences). Platelets were gated by forward-side scatter characteristics.

### In Vitro Binding Assay

Wells of 96-well Nunc MaxiSorp™ plates (Thermo Scientific) were coated overnight with 50 μl of indicated proteins, diluted in PBS at a concentration of 5 μg/ml. Plates were washed three times with Tris buffered saline (TBS) containing 0.1% Tween 20 (TBS-T) and blocked for 1.5 h at 37° C. with 2% fat free milk in TBS, 0.02% Tween 20. After one washing step, wells were incubated with the indicated anti-G6b monoclonal antibodies (10 μg/ml) or IgG control, diluted in 3% BSA in TBS-T for 1.5 h at RT. After five washing steps, wells were incubated with HRP-conjugated anti-mouse IgG antibody (Sigma) for 1 h at RT at low agitation. Plates were washed seven times and signals were developed with TMB. The reaction was stopped by addition of 2 M H₂SO₄ (50 μl/well) and absorbance at 450 nm and 570 nm (background) was measured with a Versa max plate reader (Molecular Devices).

### Immunofluorescence Staining

Glass coverslips (13 mm, round) were coated with 10 μg/ml collagen I (Takeda) overnight, 4° C. before blocking with 5 mg/ml denatured fatty acid free BSA for 1 hour, RT. Washed platelets (2×10⁷/ml) were allowed to spread for 45 minutes, 37° C., followed by fixing with 3.7% paraformaldehyde for 10 minutes, RT. Where indicated, platelets were permeabilized with 0.2% Triton-X 100 for 5 minutes, RT. Sample were stained, or not, with the anti-G6b mouse monoclonal antibody 17-4 (5 μg/ml) for 1 hour at RT, washed and stained with anti-mouse Alexa488-conjugated antibody for 1 hour, RT before mounting onto microscope slides using Hydromount (National Diagnostics). All images were captured using the 488 nm Argon laser line of a Leica SP2 inverted confocal with the 63×1.4 NA oil objective.

### Immunohistochemistry

Bone marrow biopsies were obtained from clinically affected individuals and control; bone marrow biopsy and histology were prepared using standard clinical procedures. Images were obtained with an Olympus BX43 microscope and DP25 digital camera and acquired with the Olympus CellSens Entry Imaging Software.

### EXAMPLES

### Example 1—Production of Antigen Binding Polypeptides of the Invention

His-tagged recombinant anti-G6b-B Fab fragment was produced by transient co-expression of heavy and light chains in HEK293 6E cells, and conditioned media containing expressed Fab fragment harvested by centrifugation after 7 days. Recombinant Fab fragment was isolated using IMAC (Nickel Sepharose excel, GE Healthcare LifeSciences), and further purified on a 120 ml HiLoad 16/60 Superdex 75 column (GE Healthcare LifeSciences). The size exclusion column was equilibrated and run in PBS.

### Example 2—Characterisation and Structural Study of Exemplary Antibody 17-4

### G6b-B ECD:Fab Binding Interactions

The crystal structure of the G6b:DP12:Fab complex clearly shows that almost all the interaction between the G6b-B ECD domain and the Fab molecule involve G6b-B residues Pro19 to Arg26 from its N-terminal beta-strand A in addition to residues His121 to Gly124 located at the end of the adjacent C-terminal beta-strand G (FIG. 12). The majority for these interactions are formed by all three CDR's from the VH chain and CDR1 and 3 from the VL chain with no significant interactions to G6b ECD being made by residues of the VL CDR2 loop.

Particularly important interactions are formed at the Fab antigen binding interface by Asp24 of G6b-B which forms a salt bridge to the VH residue Arg69 (CDR2) and also additional hydrogen bonds with the VH residue Ser121 (CDR3). Other notable hydrogen bonds are formed between the side chain of G6b-B's Arg26 and the main chain carbonyl of the VH CDR3 residue Tyr119 and also between main chain amide of G6b-B's Gly124 and the side chain of the VH CDR2 residue Asp71. See FIG. 12 for more visual representation of the antibody binding to the epitope.

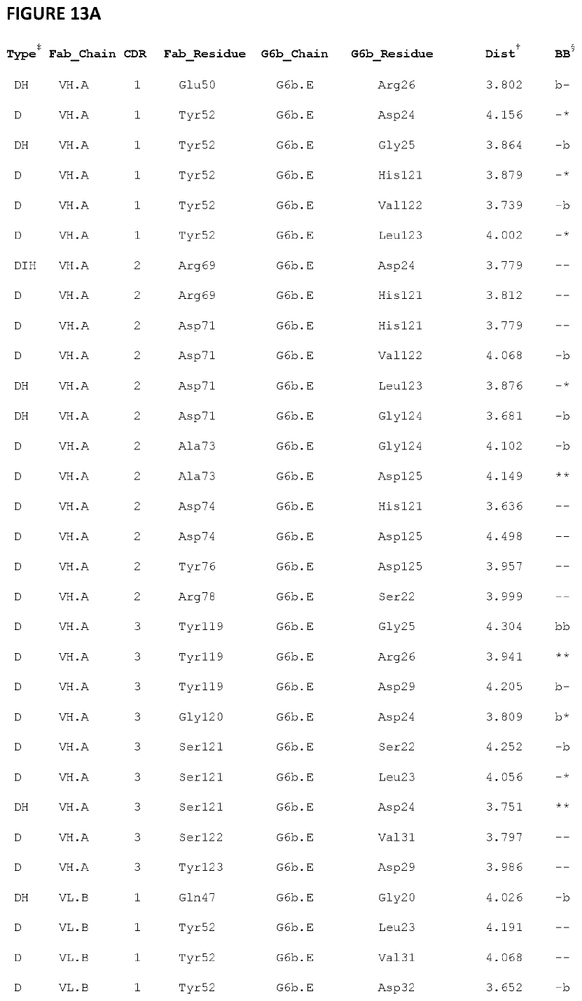

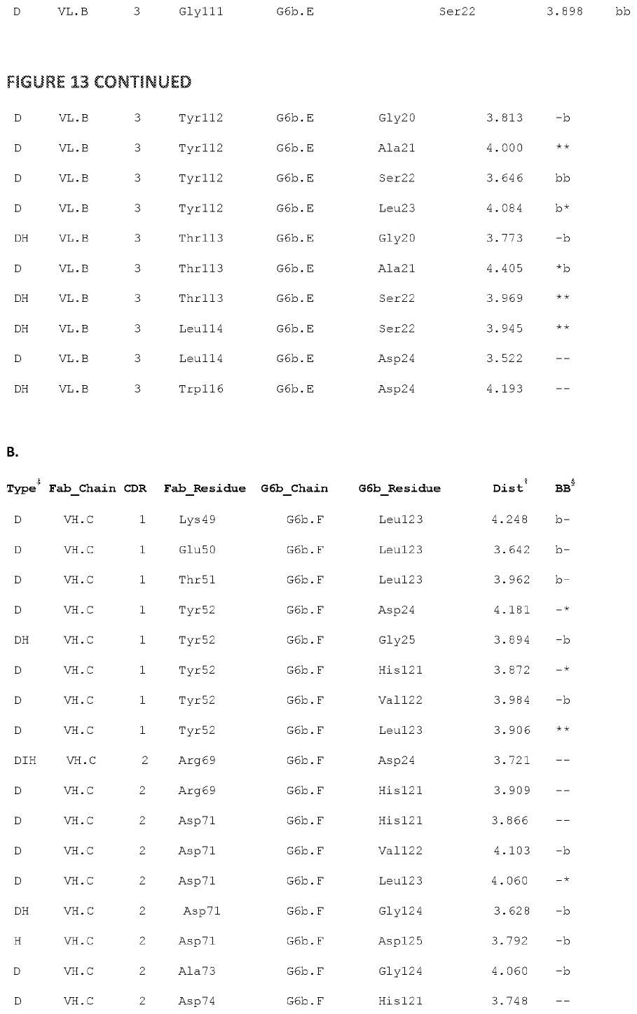

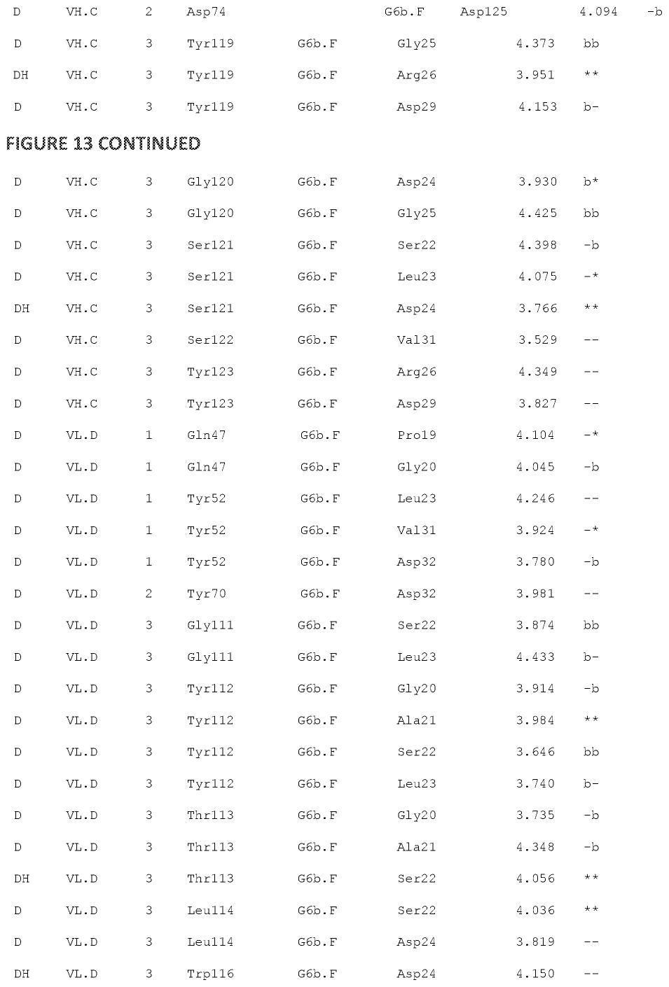

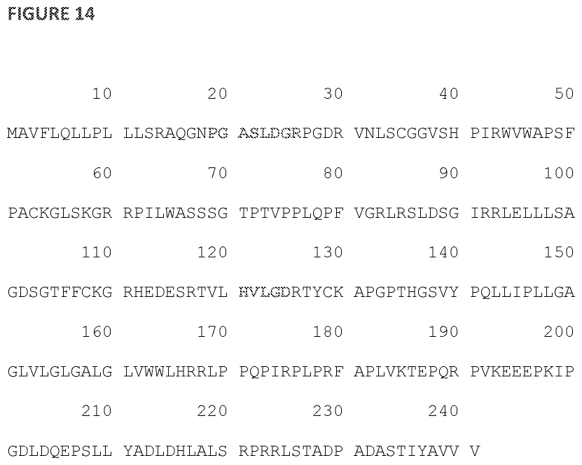

A summary of all the residue interactions formed at the binding interface between G6b-B ECD and Fab for both G6b:Fab complexes in the dimer can be found in FIGS. 11 and 12 and in tabular form in FIG. 13.

TABLE 1
Data collection and processing statistics for G6b-B ECD-Fab-DP12
Table 1 Data collection and processing statistics for G6b-B
ECD-Fab-DP12

| X-ray source | 103 (Diamond) |
|---|---|
| Wavelength [Å] | 0.97624 |
| Detector | PILATUS3.6M |
| Temperature [K] | 100 |
| Space group | C 2 |
| Cell: a; b; c; [Å] | 1 83.80; 72.34; 131.04 |
| α; β; γ; [°] | 90.0; 124.5; 90.0 |
| Resolution [Å] | 3.13 (3.18-3.13) [symbol] |
| Unique reflections | 24543 (1249) |
| Multiplicity | 3.0(3.1) [symbol] |
| Completeness [spherical %] | 97.2(98.8)² |
| Rsym [%] ³ | 15.9 (146)² |
| Rmeas [%] ⁴ | 19.3 (176)² |
| Mean(I)/sd ⁵ | 4.0 (0.7)² |
| CC(½) | 0.990 (0.348) |

¹ Diamond Light Source, Oxford, UK.
²Values in parenthesis refer to the highest resolution bin.
³ $R = \frac{{Σ}_{hkl} {Σ}_{j} \vert {I}_{hkl , j} - 〈 {I}_{hld} 〉 \vert}{{Σ}_{hkl} {Σ}_{j} {I}_{hkl , j}}$ ⁴ $R \text{?} = \frac{{Σ}_{hkl} \sqrt{\frac{\text{?}}{\text{?} - 1}} {Σ}_{j = 1}^{n} \vert {I}_{hkl , j} - 〈 {I}_{hld} 〉 \vert}{{Σ}_{hld} {Σ}_{j} {I}_{hld , j}}$ ⁵ CC_{½} = the correlation between intensities from random half-data sets. [symbol]
indicates data missing or illegible when filed

TABLE 2
Refinement statistics for G6b-B ECD-Fab-DP12¹
Table 2 Refinement statistics for G6b-B ECD-Fab-DP12¹

| | |
|---|---|
| Resolution [Å] | 63.10-3.13 |
| Number of reflections | 18509/885 |
| (working/test) | |
| Rcryst [%] | 22.6 |
| Rfree[%] ² | 26.0 |
| Total number of atoms: | |
| Protein | 7622 |
| Water | 0 |
| Heterogen (including | 230 |
| ligand) 230 | |
| Deviation from ideal | |
| geometry: ³ | |
| Bond lengths [Å] | 0.01 |
| Bond lengths [Å] | 1.18 |
| Bonded B's [Å2] ⁴ | 5.21 |
| Ramachandran plot: ⁵ | |
| Most favoured regions | 91.8 |
| [%] | |
| Additional allowed | 7.1 |
| regions [%] | |
| Disallowed regions [%] | 1.0 |

¹Values as defined in REFMAC5, without sigma cut-off
² Test-set contains 5.0% of measured reflections
³ Root mean square deviations from geometric target values
⁴ Calculated with MOLEMAN2
⁵ Calculated with COOT

### Conclusion

The X-ray crystal structure of the G6b-B ECD:Fab:DP12 complex reveals that two G6b-ECD domains form a close dimer together with a single DP12 molecule bound along a highly positively charged cleft at the dimer domain interface. It is believed that the G6b-B ECD dimerisation occurs as a direct result of DP12 binding. Furthermore a single Fab fragment molecule is observed to bind to each of the two G6b ECD domains (see FIG. 11). The crystal structure clearly reveals that the epitope recognised by the Fab CDRs consists mainly of the N-terminal β-strand of the G6b-ECD together with several residues at the C-terminal end of the adjacent β-strand.

### Example 3—Biological Functions of Exemplary Antibody 17-4

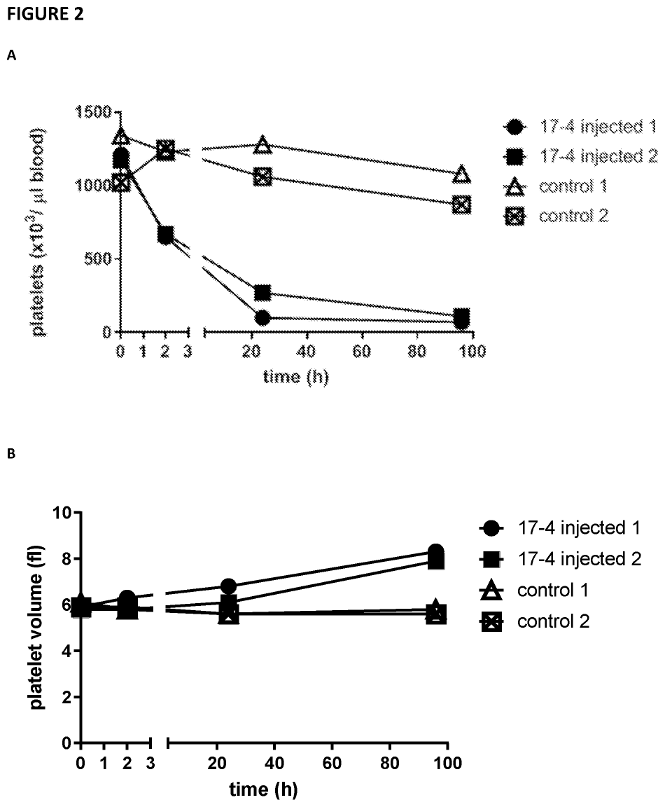

G6b-B humanised mice were treated with exemplary antibody 17-4 or PBS and platelet count and volume measured at intervals over a period of 100 hours. Mice injected with exemplary antibody 17-4 displayed a markedly reduced platelet number, and an increased platelet volume (FIG. 2).

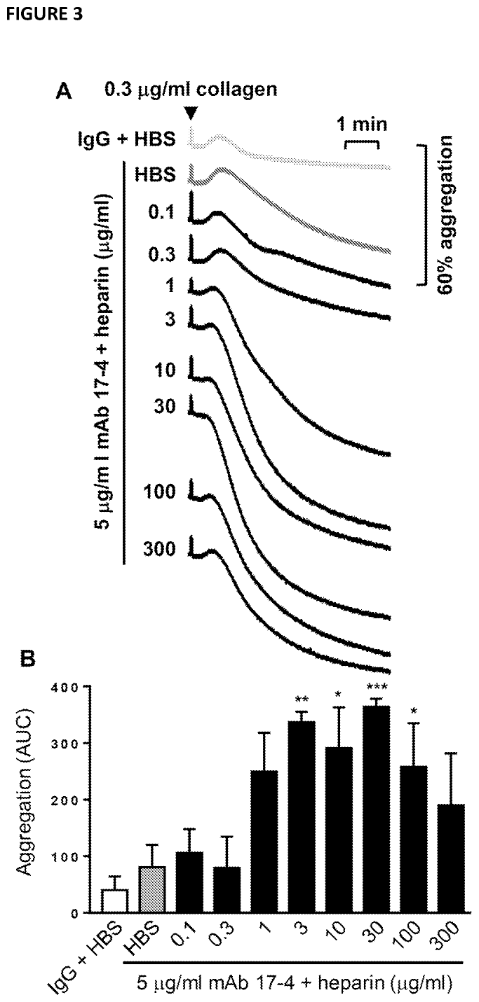

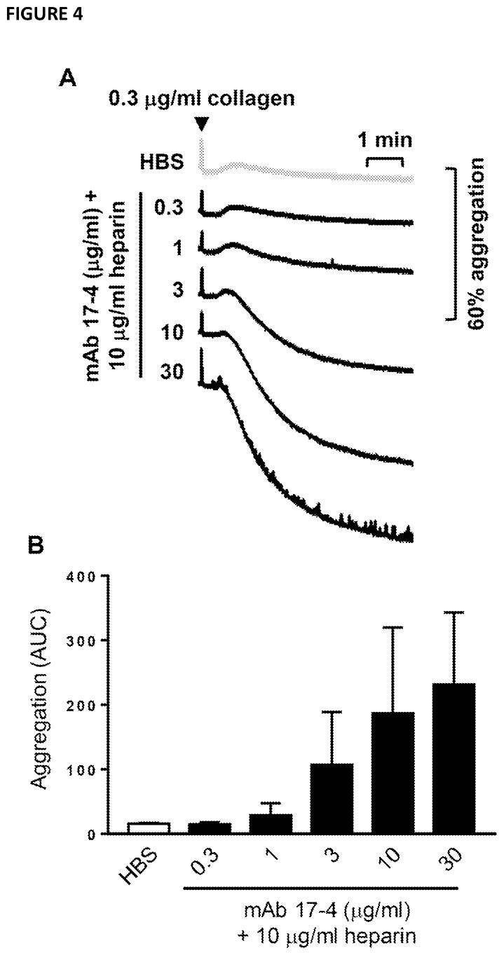

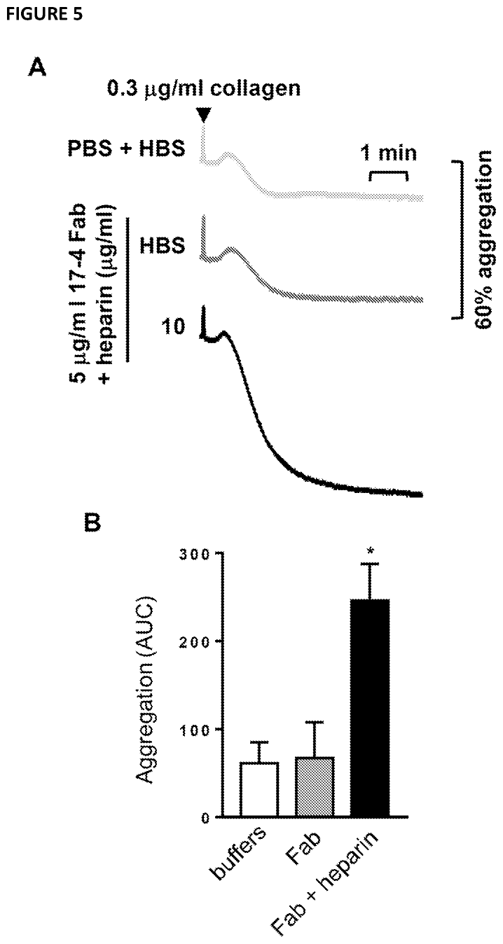

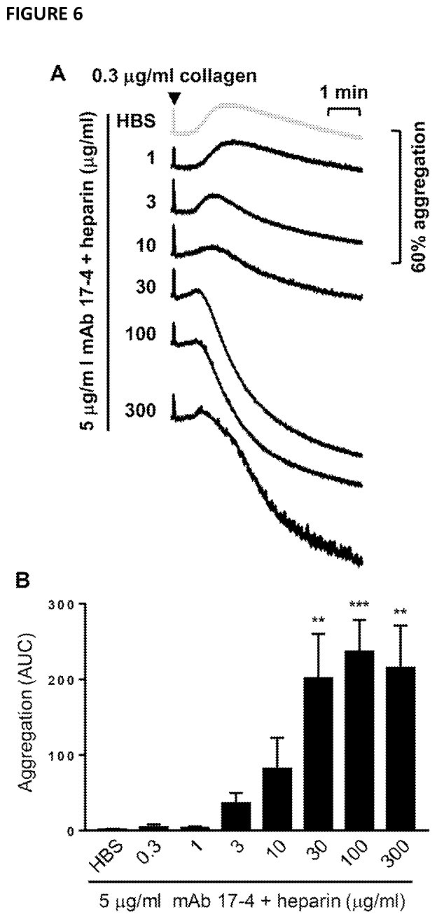

FIGS. 3, 4 and 5 illustrate that the exemplary antibody 17-4 causes an increase in heparin-mediated potentiation of human washed platelet aggregation in a dose-dependent manner. Further, a Fab fragment of the exemplary antibody 17-4 is shown in FIG. 5 to potentiate the effect of the G6b-B ligand heparin on washed platelet aggregation. FIG. 6, shows that the full monoclonal antibody of exemplary 17-4 also displays the same functional effects on platelets of G6b-B humanized mice. The exemplary antibody binds the ectodomain of G6b-B, and inhibits inhibitory signalling from G6b-B by altering the clustering of heparin ligand-induced G6b-B dimers. This may result in increased signalling from activatory receptors that G6b-B normally suppresses, thus altering platelet reactivity systemically and platelet production in the bone marrow. This provides new alternative ways to treat diseases and disorders associated with platelet homeostasis.

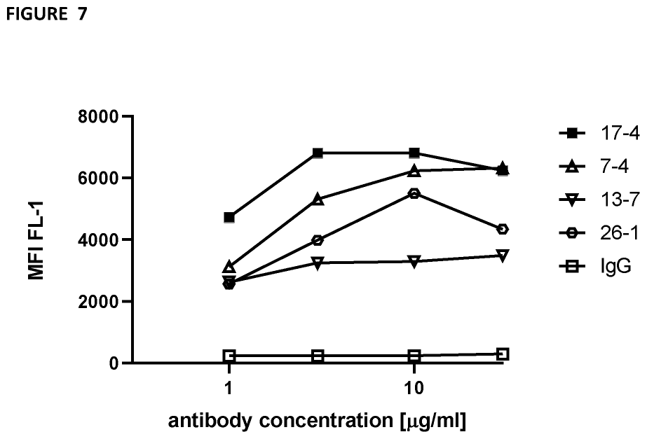

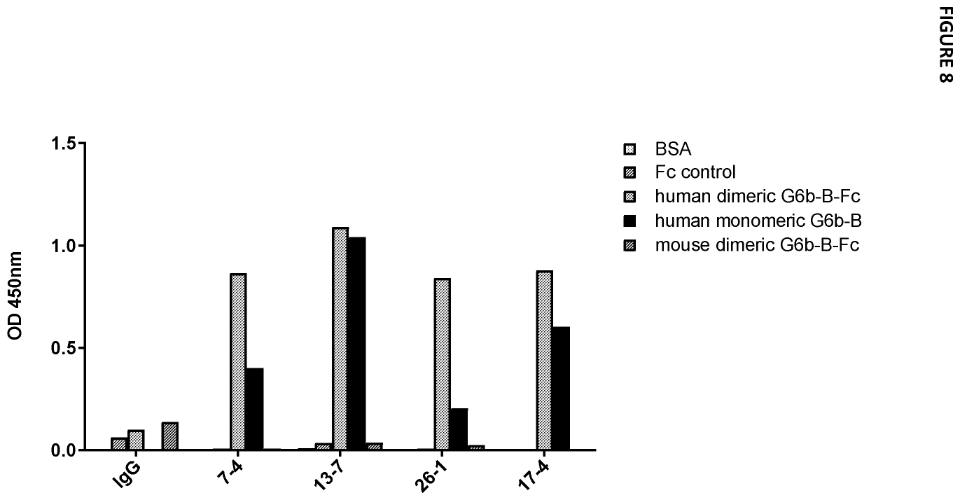

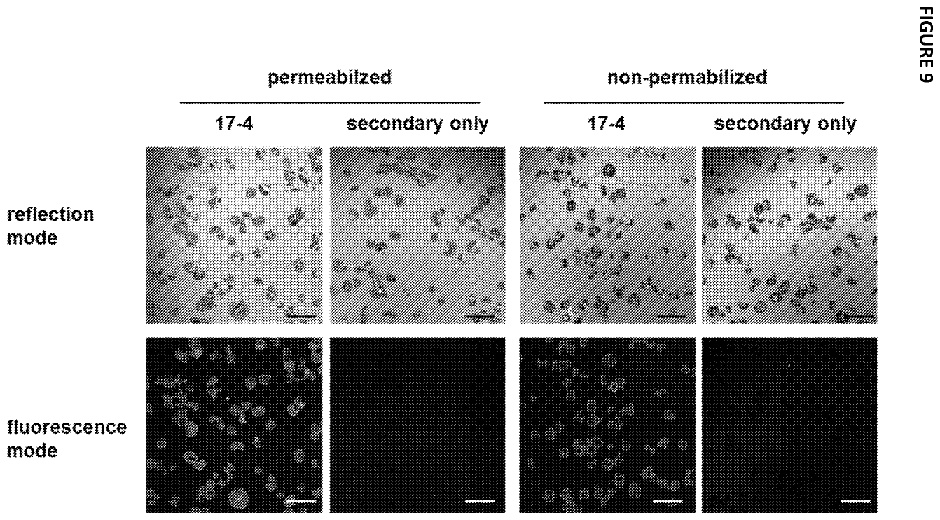

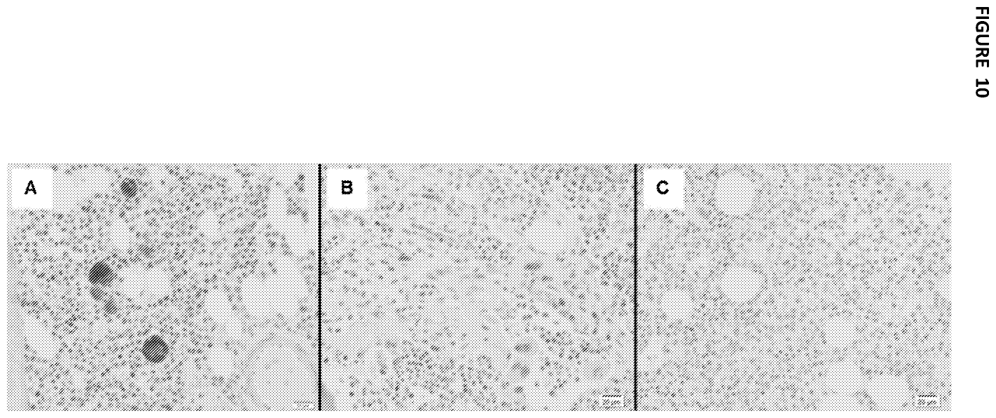

Exemplary antibody 17-4 also demonstrates strong binding to human platelets, which have the cell surface receptor G6b-B (FIG. 7), and platelets from humanized G6b-B mice, that express only the human orthologue of the receptor. The antibody also binds strongly to both human recombinant monomeric and dimeric G6b-B, but not to mouse G6b-B (FIG. 8). These results illustrate the specificity of the 17-4 antibody.

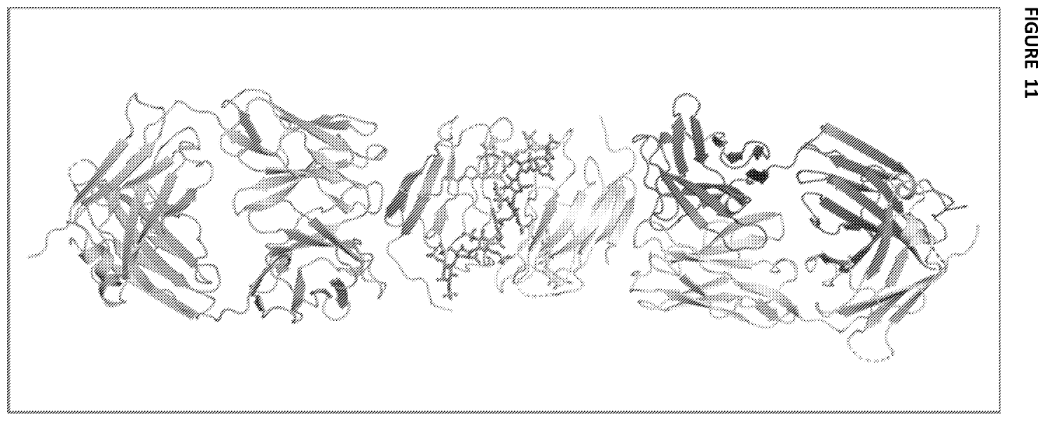

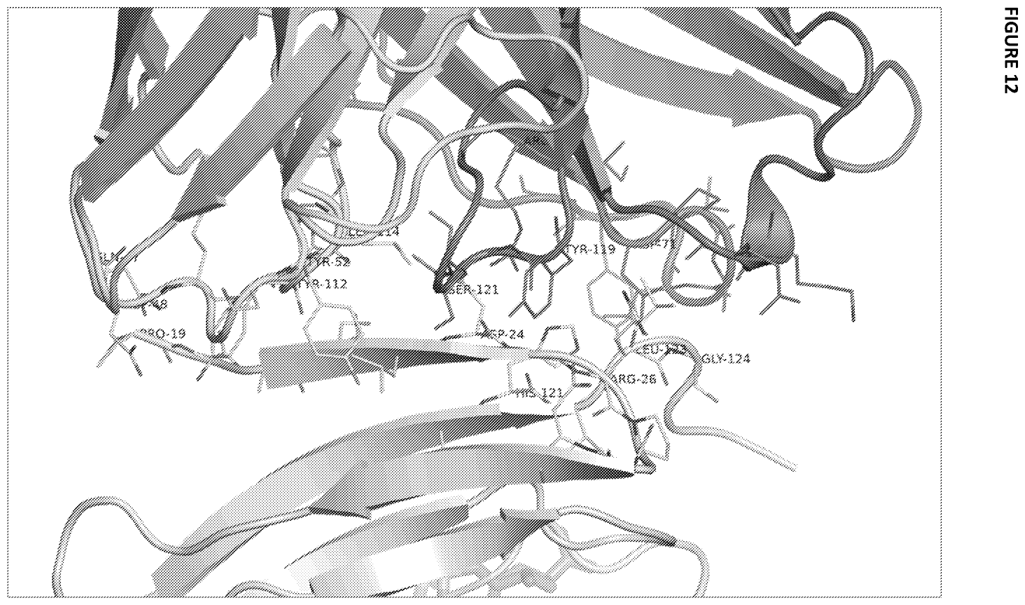

The exemplary 17-4 antibody binds specifically to G6b-B on platelets as illustrated in FIG. 9 using immunofluorescence, as well as to G6b-B on megakaryocytes in the bone marrow as illustrated in FIG. 10.

More detailed visual representation of the 17-4 Fab: G6b-B complex, with and without heparin ligand bound, can be found in the ribbon illustrations in FIGS. 11 and 12. The crystal structure demonstrates that the epitope and ligand binding site are distinct, which provides an ideal binding site for an antibody without interfering with ligand engagement. This therefore can be advantageous in enhancing biological effects of ligands.

### Example 4—Determination of the Binding Affinity of Exemplary Antibody 17-4 to G6b-B

17-4 Fabs produced according to the method described above, were demonstrated to bind to G6b-B monomers and dimers in a two stage reaction mechanism, resulting in a specific, high affinity interaction (see Table 2).

TABLE 3
antibody binding affinity of 17-4 Fabs to monomeric or dimeric human G6b-B

| | | | | | | Rmax | |
|---|---|---|---|---|---|---|---|
| Analyte | Channel | kon 1 | koff 1 | Kon 2 | koff 2 | (RU) | KD |
| Fab | G6b-B | 4.95E+04 | 5.32E−03 | 8.24E−03 | 9.68E−06 | 1059.7 | 1.26E−10 |
| | monomer | | | | | | |
| Fab | G6b-B | 3.12E+04 | 4.80E−03 | 7.80E−03 | 1.33E−05 | 1971.3 | 2.62E−10 |
| | dimer | | | | | | |

1. An isolated antigen binding molecule or fragment thereof which specifically binds to human G6b-B.

2. An isolated antigen binding molecule or fragment thereof which competes for binding to human G6b-B with the antigen binding polypeptide or fragment thereof according to claim 1.

3. The isolated antigen binding molecule or fragment thereof according to claim 1 or 2, which specifically binds to an epitope on human G6b-B defined by residues equivalent to at least Asp 24, Arg 26 and Gly 124 on human G6b-B.

4. The isolated antigen binding molecule or fragment thereof according to any of claims 1-3, which specifically binds to an epitope on human G6b-B defined by residues equivalent to at least Pro19 to Arg26 and His121 to Gly124.

5. The isolated antigen binding molecule or fragment thereof according to any one of claims 1-4, which is an antibody or fragment thereof.

6. The antibody or fragment thereof according to claim 5, which is a monoclonal antibody, bispecific antibody, Fab, (Fab′)2, scFv, Fv, dAb, Fd or a diabody.

7. The antibody or fragment thereof according to claim 5, which is a monoclonal antibody.

8. The isolated antigen binding molecule or fragment thereof according to any one of claims 1-7, comprising an antigen binding portion which comprises heavy and/or light chain variable regions which comprise one or more of the CDRs defined by SEQ ID NOs: 1-6 or a sequence having at least 80% identity thereto; or
a variant of one or more of the CDR sequences defined by Seq ID No: 1, 2, 3, 4, 5 and 6 having one, two or three amino acid variations from the recited CDR sequence provided that the antigen binding molecule or fragment thereof retains the ability to bind to G6b-B.

9. The isolated antigen binding molecule or fragment thereof according to any one of claims 1-8, comprising an antigen binding portion comprising a heavy and/or light chain variable region, wherein:
a) the heavy chain variable region comprises one or more of the hypervariable regions comprising sequences of at least 80% identity to:
CDR1 of SEQ ID NO: 1
CDR2 of SEQ ID NO: 2
CDR3 of SEQ ID NO: 3 or direct CDR equivalents thereof; and/or
b) the light chain variable region comprises one or more of the hypervariable regions comprising sequences of at least 80% identity to:
CDR1 of SEQ ID NO: 4
CDR2 of SEQ ID NO: 5
CDR3 of SEQ ID NO: 6 or direct CDR equivalents thereof.

10. The isolated antigen binding molecule or fragment thereof according to any one of claims 1-9, comprising an antigen binding portion comprising a heavy and/or light chain variable region, wherein:
a) the heavy chain variable region comprises the hypervariable regions:
CDR1 of SEQ ID NO: 1
CDR2 of SEQ ID NO: 2
CDR3 of SEQ ID NO: 3
or sequences of at least 80% identity thereto;
and/or
b) the light chain variable region comprises the hypervariable regions:
CDR1 of SEQ ID NO: 4
CDR2 of SEQ ID NO: 5
CDR3 of SEQ ID NO: 6
or sequences of at least 80% identity thereto.

11. The isolated antigen binding molecule or fragment thereof according to any one of claims 1-10, comprising a variable heavy chain region with at least 80% sequence identity to SEQ ID NO: 7 and a variable light chain region with at least 80% sequence identity to of SEQ ID NO: 8.

12. The isolated antigen binding molecule or fragment thereof according to any one of claims 1-11, comprising a variable heavy chain region of SEQ ID NO: 7 and a variable light chain region of SEQ ID NO: 8.

13. The isolated antigen binding molecule or fragment thereof according to any one of claims 1-12, which binds to human G6b-B with an affinity (KD) of about 1.0×10-10 M and 3.1×10-10 M.

14. The isolated antigen binding molecule or fragment thereof according to claim 13, which binds to monomeric human G6b-B with an affinity (KD) of about 1.0×10-10 M to about 1.5×10-10 M, and/or:
which binds to dimeric human G6b-B with an affinity (KD) of about 2.0×10-10 M to about 1.5×10-10 M.

15. A nucleic acid encoding the antigen binding molecule or fragment thereof according to any one of claims 1-14.

16. The nucleic acid according to claim 15, which encodes the heavy chain region defined by SEQ ID NO: 9 and/or the light chain region defined by SEQ ID NO: 10.

17. A vector comprising the nucleic acid of claim 11 or 12.

18. A host cell comprising the vector according to claim 12.

19. The host cell according to claim 18, which is a mammalian cell.

20. The host cell according to claim 19, which is a HEK293 cell, or derivative thereof.

21. A pharmaceutical composition comprising the isolated antigen binding molecule or fragment thereof, nucleic acid, vector and/or host cell of any one of claims 1-20, optionally further comprising one or more pharmaceutically acceptable excipient or diluents.

22. The pharmaceutical composition according to claim 21, which is intended to be administered intravenously.

23. The isolated antigen binding molecule or fragment thereof, nucleic acid, vector, host cell, or pharmaceutical composition of any one of claims 1-22 for use in treating a disease or disorder associated with dysregulated platelet homeostasis.

24. Use of the isolated antigen binding molecule or fragment thereof, nucleic acid, vector, host cell, or pharmaceutical composition of any one of claims 1-22 in a method of treating diseases or disorders associated with dysregulated platelet homeostasis.

25. Use of the isolated antigen binding molecule or fragment thereof, nucleic acid, vector, host cell, or pharmaceutical composition of any one of claims 1-22 in the manufacture of a medicament for the diagnosis or treatment of diseases or disorders associated with dysregulated platelet homeostasis.

26. The isolated antigen binding molecule or fragment thereof, nucleic acid, vector, host cell, or composition for use according to claim 23 or use of the isolated antigen binding molecule or fragment thereof, nucleic acid, vector, host cell, or pharmaceutical composition according to claim 24 or 25, wherein the disease or disorder is one or more of a myeloproliferative disease, or a disease or disorder caused as a secondary effect of a subject having a myeloproliferative disease, or a myeloproliferative neoplasm, thrombocythemia, myelofibrosis, thrombocytosis, thrombocytopenia (for example macrothrombocytopenia, microthrombocytopenia and normothrombocytopenia), haemophilia, Bernard-Soulier Syndrome, Glanzmann thrombasthenia, alpha granule deficiency, delta storage pool deficiency, Scott syndrome, myelodysplastic syndromes.

27. The isolated antigen binding molecule or fragment thereof, nucleic acid, vector, host cell, or composition for use, or use of the isolated antigen binding molecule or fragment thereof, nucleic acid, vector, host cell, or pharmaceutical composition according to any one of claims 23-26,
wherein the isolated antigen binding molecule or fragment thereof, nucleic acid, vector, host cell, or pharmaceutical composition is suitable to be administered with another therapeutic.

28. The isolated antigen binding molecule or fragment thereof, nucleic acid, vector, host cell, or composition for use according to claim 27, wherein the another therapeutic is heparin or a derivative thereof, radiotherapy or a chemotherapeutic agent.

29. A method of modulating platelet homeostasis in a subject, comprising contacting a cell with, or administering to the subject, an effective amount of an isolated antigen binding molecule or fragment thereof, nucleic acid, vector, host cell, or composition according to any one of claims 1-21.
